(19) United States
(12) Patent Application Publication
(10) Pub. No.: US 2004/0033949 A1
(43) Pub. Date: Feb. 19, 2004

(54) Use of VEGF for treating bone defects

(75) Inventors: Stuart Bunting, Half Moon Bay, CA (US); Richard Carano, San Ramon, CA (US); Ellen Hope Filvaroff, San Francisco, CA (US); Richard Andre Gosselin, El Granada, CA (US); Franklin V. Peale JR., San Carlos, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/431,105

(22) Filed: May 6, 2003

Related U.S. Application Data
(60) Provisional application No. 60/378,275, filed on May 6, 2002.

(51) Int. Cl. A61K 38/18; A61K 9/22

(52) U.S. Cl. 514/12; 424/468

(57) ABSTRACT

The present invention provides pharmaceutical compositions comprising VEGF or variants thereof for promoting bone formation, in vitro and in vivo. Methods of using those compositions are also provided. Compositions and methods of the present invention can be used for promoting and improving the repair process in subjects with bone defects.

This application claims priority under 35 U.S.C. 119(e) to the U.S. provisional application Ser. No. 60/378,275, filed May 6, 2002, the disclosure of which is expressly incorporated herein by reference in its entirety.

### FIELD OF THE INVENTION

This invention relates to effective treatments of bone defects using biologically active compositions, especially VEGF and variants thereof.

### BACKGROUND OF THE INVENTION

Bone is a dynamic biological tissue composed of metabolically active cells that are integrated into a rigid framework. It is under a continuously occurring state of bone deposition, resorption and remodeling, processes that enable and facilitate bone regeneration and repair. The cellular components of bone consist of osteogenic precursor cells, osteoblasts, osteoclasts, osteocytes and the hematopoietic elements of bone marrow. Osteoblasts are mature, metabolically active, bone-forming cells. They secret osteoid, the unmineralized organic matrix that subsequently undergoes mineralization, giving the bone its strength and rigidity. Osteoblasts also play a role in the activation of bone resorption by osteoclasts. Osteoclasts are multinucleated, bone-resorbing cells controlled by hormonal and cellular mechanisms. These cells function in groups termed “cutting cones” that attach to bare bone surfaces and, by releasing hydrolytic enzymes, dissolve the inorganic and organic matrices of bone and calcified cartilage. This process results in the formation of shallow erosive pits on the bone surface called Howship lacunae. For general descriptions of bone anatomy and histology, see, for example, Copenhaver et al (1978): The connective tissues: cartilage and bone, in Copenhaver et al. (eds.): BAILEY'S TEXTBOOK OF HISTOLOGY, ed. 17.170-205 (Baltimore:Williams & Wilkins); Dee (1988): Bone Healing, in Dee et al (eds.): PRINCIPLES OF ORTHOPAEDIC PRACTICE, 68-73 (NY:McGraw-Hill); and Recker (1992): Embryology, anatomy, and microstructure of bone, in Coe and Favus (eds.): DISORDERS OF BONE AND MINERAL METABOLISM, 219-240 (NY:Raven).

Bone metabolism is under constant regulation by a host of honnonal and local factors. The best known of these factors are the bone morphogenetic proteins (BMPs). BMPs are members of the transforming growth factor (TGF)-β superfamily, a large family of secreted signaling molecules. Wozney and Rosen (1998) Clin. Orhtop. Rel. Res. 346:26-37. Several members of this gene family have been identified. The major function of BMPs is to induce new bone formation. Numerous studies have demonstrated that BMPs can efficiently heal large bony defects as well as segmental defects. The proteins that have received much of the attention are BMP-2 and BMP-7 (or osteogenic protein-1, OP-1). Niyibizi and Kim (2000) Exp. Opin. Invest. Drugs 9:1573-1580. Recombinant proteins have now been generated and are undergoing clinical trials. BMP-2, when combined with inactivated demineralised bone matrix used as a carrier, has been shown to induce de novo cartilage and bone formation in rat, sheep and dog bone defect models. Lee et al. (1994) J. Biomed. Mater. Res. 28:1149-1156; Cook et al. (1995) J. Bone Joint Surg. 77A:734-750. BMP-7 has also been shown to heal large segmental defects in animal models. Cook et al. (1994) J. Bone Joint Surg. 76A:827-838.

A variety of pathological conditions are characterized by the need for enhanced bone formation that occurs as a result of trauma, where sufficient osteogenic activity is crucial for proper and complete restoration of the damaged bone structure. Bone repair is a complex, multi-step process involving proliferation, migration, differentiation, and activation of a number of cell types. In a manner similar to that of normal bone development, bone formation during the healing of fracture can occur through two distinct physiological processes. If bone segments are stabilized, or during development of some skull and facial bones and parts of the mandible and clavicle, mesenchymal precursor cells differentiate directly into bone-forming osteoblasts in a process called intramembranous ossification. Alternatively, in a biomechanically unstable environment, or in development of long bones of the appendicular skeleton and vertebrae of the axial skeleton, bone formation occurs via a cartilage intermediate in a process called endochondral ossification. Mandracchia et al. (2001) Clin. Pod. Med. Surg. 18:55-77; Gittens et al. (2001) J Drug Targeting 9:407-429.

It has been suggested that a crucial event during the process of fracture healing is angiogenesis, during which vascular endothelial cells proliferate, prune and reorganize to form new vessels from preexisting vascular network. Spector et al. (2000) Am. J. Physiol. Cell Physiol. 280:72-80. Many studies have shown increased vascularity and blood flow that occurring at the site of a fracture, yet the precise mechanism(s) whereby vascularization regulates ossification remains unclear.

Expression of particular growth factors—such as basic fibroblast growth factor (bFGF), platelet derived growth factor (PDGF), transforming growth factor beta (TGF-β), vascular endothelial growth factor (VEGF), and bone morphogenetic proteins (BMP)—during the course of healing suggests a possible regulatory role for these secreted factors in bone repair.

Indeed, each of these factors, except VEGF, have been shown to stimulate bone healing to varying degrees in distinct animal models.

VEGF, a potent mitogen for vascular endothelial cells, has been reported as an important factor for hypertrophic cartilage structure and vascularity within the developing growth plate of long bones. Gerber et al. (1999) Nat. Med. 5:623-628. Whether VEGF is critical for bone repair, especially during intramembranous ossification, has not yet been determined. VEGF is expressed in the fracture callus in animal models in much the same temporal and spatial pattern as during long bone development. Other pro- and anti-angiogenic factors expressed in the growth plate of developing bones are also present in the fracture callus during repair. Thus, the fracture callus contains many factors which may promote appropriate bone healing by coordinating angiogenesis with bone homeostasis. Tatsuyama et al. (2000) Eur. J Histochem. 44: 269-278; Gerber & Ferrara (2000) Trends Cardiovasc. Med. 10:223-228; Hadjiargyrou et al. (2000) J. Bone Miner. Res. 15:1014-1023; Glowacki (1998) Clin. Orthop. 355:S82-S89.

Incomplete or inappropriate repair could lead to various pathological conditions of the bone, including but not limited to, osteoporosis, osteonecrosis, or aberrant or delayed fracture healing. It is estimated that 5.6 million fractures occur annually in the United States alone, and, despite advances in surgical techniques, about 5-10% of these result in delayed or impaired healing, known as delayed unions or non-unions. Furthermore, the majority of non-unions (up to 80%) are atrophic, i.e. avascular. Einhorn (1995) J. Bone Joint Surg. 77:940-956. Failure of proper and complete fracture healing results in pain, instability, and associated loss of functions of the suffering limb. Risk factors for impaired bone healing include periosteal disruption, iatrogenic events, infection, compound injuries, smoking, drug use, systemic disorders such as diabetes and poor nutrition, and vascular disruption concomitant with bone injury. In addition, because a significant number of fractures occur in productive individuals young and old, the degree of disability caused by this problem is substantial. Thus, enhancement of the fracture repair process would be of great benefit to ensure the rapid restoration of skeletal function. The ability of injured patients to return to the work force or to recreational activities early and fully would not only have a tremendous economic impact on society but would also improve the overall physical and mental well being of the patients.

### SUMMARY OF THE INVENTION

The present invention provides methods for effectively promoting bone formation using a composition comprising VEGF or variants thereof. The methods described and contemplated herein can be used to treat various bone defects where sufficient bone formation is required. Preferably, the methods are used to promote bone repair in a fracture caused by trauma. In one aspect, the methods of the invention are used to improve delayed unions or to treat nonunions. Other bone defects that can be treated by the methods of the invention include, but not limited to, vertebral body or disc injury/destruction, spinal fusion, injured meniscus, avascular necrosis, cranio-facial repair/reconstruction, cartilage destruction/damage, osteoarthritis, osteosclerosis, osteoporosis, implant fixation, and other inheritable or acquired bone disorders and diseases.

In a preferred embodiment, the composition comprising VEGF or variant thereof is administered through a local delivery system. In one aspect, the composition is in a slow release preparation. One slow release preparation as an example comprises polylactic acids in combination with a hydrophilic solvent such as benzyl alcohol and a hydrophobic solvent such as benzyl benzoate. Also contemplated is a slow release composition comprising polylactic acid, VEGF and at least one solvent.

In another aspect, VEGF is locally administered via a gene delivery system comprising a plasmid encoding VEGF. The gene delivery system can be a viral vector such as retroviral, adenoviral, AAV or lentiviral vector. Alternatively, the gene delivery system can be a nonviral system encapsulating the plasmid, such as a lipid preparation.

Also provided by the present invention are methods of promoting bone formation using VEGF in serial or in combination with other osteogenic factors. Factors known to be involved in promoting bone formation include, but not limited to, BMPs, FGF, growth hormone, PDGF, TGF-β, GDF-5 and OP-1. Moreover, the above described therapeutic compositions can be used together with a mechanical device to treat or alleviate bone injuries.

An article of manufacture comprising the above described compositions and an instruction for using the same is also provided.

### BRIEF DESCRIPTION OF DRAWINGS

FIGS. 1A-1D show that Flt-IgG treatment impairs fracture repair. (A, B) Percent calcified callus for control (Con), Flt-IgG (Flt), or IgG (IgG) treated mice at 7 days (A) or 14 days (B) after fracture. (C, D) Mean mineral density (MMD) of total (Total) or calcified (Calcified) callus at 7 (C) or 14 (D) days. Shown are means±SEM. * represents p<0.05; ** represents p<0.01 relative to IgG treated mice.

FIGS. 2A-2D show that Flt-IgG treatment impairs repair of cortical bone defects. (A, B) Degree of mineralization in defect sites of control (Con), Flt-IgG (Flt), or IgG (IgG) treated mice at 7 (A) or 14 (B) days after injury. (C, D) MMD of total (Total) or calcified (Calcified) callus at 7 (C) or 14 (D) days after injury. Shown are means±SEM. * represents p<0.05; ** represents p<0.01 relative to IgG treated mice.

FIGS. 3A-3C show that Flt-IgG treatment decreases angiogenesis. PECAM measurements of the (A) bone (femur) from the fractured leg (right) and the contralateral control leg (left), or the (B) tissue from the left (L) normal and right(R) fractured leg of control (Con), Flt-IgG (Flt), or IgG (IgG) treated mice at 7d post-fracture. * shows statistically significant (p<0.05) induction of vascularity in the right, fractured leg, relative to the left, intact leg in each treatment group. (C) Percent increase in vascularity in the fractured leg relative to the contralateral control was calculated as (PECAM_{right}−PECAM_{left})/PECAM_{left}. Shown are means±SEM. In (A) and (C)* represents statistically significant (p<0.05) differences between Flt-IgG treated mice and both control and IgG treated mice.

FIGS. 4A-4D depict that VEGF initiates a positive autocrine loop in osteoblasts. (A) Nodule formation or (B) alkaline phosphatase activity were measured in primary human osteoblasts treated with VEGF (0, 1, 5, 10, 25, or 50 ng/ml) or anti-VEGF (αV). (C) VEGF or (D) bFGF production by primary human osteoblasts under 21% 02, i.e. normoxia (N) or 2% O₂, i.e. hypoxia (H) at 6 (6 h), 12 (12 h), 24 (24 h), 36 (36 h), or 48 (48 h) after cells adherence. Shown are means±SEM.

FIGS. 5A-5D show that local VEGF treatment promotes fracture repair. (A) Percent calcified callus in mice treated with VEGF (10 ug) (V) or carrier alone (−). (B) Mean mineral density of the total (Total) or calcified (Calcified) callus of mice treated with VEGF (10 ug) (V) or carrier alone (−). (C, D) PECAM measurements in femurs (Femur) (C) or tibiae (Tibia) (D) or tissues (Tissue) of the right, fractured leg (R) or contra-lateral, control left leg (L) of mice treated with VEGF (10 ug) (V) or carrier alone (−) at 7 days post-fracture. Shown are means±SEM. *=p<0.05 and **=p<0.005 of VEGF-treated (V) relative to carrier alone (−).

FIGS. 6A-6C show that VEGF stimulates repair of a segmental gap defect. (A) 3D renderings of μCT images of the rabbit radius critical defect model (1 cm gap) at 28 days after surgery in control (Placebo) and VEGF (250 μg) treated (VEGF) animals. The gap in the VEGF treated bone represents the site of the pump catheter. (B) Volumes of the total (Total Callus) or calcified (Calcified Callus) callus in rabbits treated with VEGF (0, 100, 250, or 1000 μg). Shown are means±SEM. * represents statistical significance (p<0.05) relative to control (0).

FIGS. 7A-7B show that receptor-selective VEGF variants promote fracture repair. 7A shows percent calcified callus and percent calcified callus minus cortex (cmc) in mice treated with carrier alone (PLAD), VEGF, Flt-selective VEGF variant (Flt-sel) or KDR-selective VEGF variant (KDR-sel). 7B shows mean mineral densities (Mean) of the total callus, the calcified (Cal.) callus, the cmc and the calcified cmc of mice treated with carrier alone or various agents.

### DETAILED DESCRIPTION OF THE PREFERRED EMBODIMENTS

The present invention provides a novel system by which locally administered VEGF or variant thereof promotes bone growth in a concerted fashion. Not wishing to be bound to particular mechanisms of action, the local VEGF therapy system provided herein may have the advantage over other known treatments in its ability to couple activities of endothelial cells (angiogenesis) with that of bone cells (osteoblasts and osteoclasts). In addition, VEGF may act as a key mediator for other angiogenic and osteogenetic factors.

In one aspect, the description provided herein demonstrates that local administration of exogenous VEGF or variant thereof enhances bone formation in subjects with bone defects, including mice with fractures and rabbits with critical size defects. Of significance are the results showing that VEGF or variant thereof, in the absence of any additional scaffold or progenitor cells, is sufficient to stimulate bone formation in two distinct bone defect models and two different species. The finding that early treatment with VEGF resulted in enhanced bone repair is consistent with the normal pattern of healing in which active vascularization occurs early (within the first week) following bone injury. The results herein show that slow-released VEGF or VEGF variant can be an effective therapeutic agent for bone injuries and that a patient with bone damage who has alterations in VEGF regulation and/or responsiveness may have a relatively poor prognosis. Accordingly, older patients and those with periosteal damage or other risk factors for delayed healing can benefit greatly from the VEGF treatment contemplated herein. In addition to fracture repair, the compositions and pharmaceutical formulations comprising VEGF or variant thereof are useful in other indications such as vertebral body or disc injury/destruction, spinal fusion, injured meniscus, avascular necrosis, cranio-facial repair/reconstruction, cartilage destruction/damage, osteoarthritis, osteosclerosis, osteoporosis, implant fixation, and other inheritable or acquired bone disorders and diseases.

Compositions of the Invention and Their Productions

Vascular endothelial cell growth factor (VEGF), a potent mitogen for vascular endothelial cells, has been reported as a key regulator of angiogenesis and vasculogenesis. Ferrara and Davis-Smyth (1997)Endocrine Rev. 18:4-25; Ferrara (1999) J. Mol. Med. 77:527-543. Compared to other growth factors that contribute to the processes of vascular formation, VEGF is unique in its high specificity for endothelial cells within the vascular system. Recent evidence indicates that VEGF is essential for embryonic vasculogenesis and angiotenesis. Carmeliet et al. (1996) Nature 380:435-439; Ferrara et al. (1996) Nature 380:439-442. Furthermore, VEGF is required for the cyclical blood vessel proliferation in the female reproductive tract and for bone growth and growth plate cartilage formation. Ferrara et al. (1998) Nature Med. 4:336-340; Gerber et al. (1999) Nature Med. 5:623-628.

In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx. Ferrara and Davis-Smyth (1997), supra. Moreover, recent studies have reported mitogenic effects of VEGF on a few non-endothelial cell types, such as retinal pigment epithelial cells, pancreatic duct cells and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

Substantial evidence also implicates VEGF's critical role in the development of conditions or diseases that involve pathological angiogenesis. The VEGF mRNA is over expressed by the majority of human tumors examined (Berkman et al. J Clin Invest 91:153-159 (1993); Brown et al. Human Pathol. 26:86-91 (1995); Brown et al. Cancer Res. 53:4727-4735 (1993); Mattern et al. Brit. J. Cancer. 73:931-934 (1996); and Dvorak et al. Am J. Pathol. 146:1029-1039 (1995)). Also, the concentration of VEGF in eye fluids are highly correlated to the presence of active proliferation of blood vessels in patients with diabetic and other ischemia-related retinopathies (Aiello et al. N. Engl. J. Med. 331:1480-1487 (1994)). Furthermore, recent studies have demonstrated the localization of VEGF in choroidal neovascular membranes in patients affected by AMD (Lopez et al. Invest. Ophtalmo. Vis. Sci. 37:855-868 (1996)). Anti-VEGF neutralizing antibodies suppress the growth of a variety of human tumor cell lines in nude mice (Kim et al. Nature 362:841-844 (1993); Warren et al. J. Clin. Invest. 95:1789-1797 (1995); Borgström et al. Cancer Res. 56:4032-4039 (1996); and Melnyk et al. Cancer Res. 56:921-924 (1996)) and also inhibit intraocular angiogenesis in models of ischemic retinal disorders (Adamis et al. Arch. Ophthalmol. 114:66-71 (1996)). Therefore, anti-VEGF monoclonal antibodies or other inhibitors of VEGF action are promising candidates for the treatment of solid tumors and various intraocular neovascular disorders.

Human VEGF was obtained by first screening a cDNA library prepared from human cells, using bovine VEGF cDNA as a hybridization probe. Leung et al. (1989) Science, 246:1306. One cDNA identified thereby encodes a 165-amino acid protein having greater than 95% homology to bovine VEGF; this 165-amino acid protein is typically referred to as human VEGF (hVEGF) or VEGF₁₆₅. The mitogenic activity of human VEGF was confirmed by expressing the human VEGF cDNA in mammalian host cells. Media conditioned by cells transfected with the human VEGF cDNA promoted the proliferation of capillary endothelial cells, whereas control cells did not. Leung et al. (1989) Science, supra.

Although a vascular endothelial cell growth factor could be isolated and purified from natural sources for subsequent therapeutic use, the relatively low concentrations of the protein in follicular cells and the high cost, both in terms of effort and expense, of recovering VEGF proved commercially unavailing. Accordingly, further efforts were undertaken to clone and express VEGF via recombinant DNA techniques. (See, e.g., Ferrara (1995) Laboratory Investigation 72:615-618 (1995), and the references cited therein).

VEGF is expressed in a variety of tissues as multiple homodimeric forms (121, 145, 165, 189, and 206 amino acids per monomer) resulting from alternative RNA splicing. VEGF₁₂₁ is a soluble mitogen that does not bind heparin; the longer forms of VEGF bind heparin with progressively higher affinity. The heparin-binding forms of VEGF can be cleaved in the carboxy terminus by plasmin to release a diffusible form(s) of VEGF. Amino acid sequencing of the carboxy terminal peptide identified after plasmin cleavage is Arg₁₁₀-Ala₁₁₁. Amino terminal “core” protein, VEGF (1-110) isolated as a homodimer, binds neutralizing monoclonal antibodies (such as the antibodies referred to as 4.6.1 and 3.2E3.1.1) and soluble forms of VEGF receptors with similar affinity compared to the intact VEGF₁₆₅ homodimer.

Several molecules structurally related to VEGF have also been identified recently, including placenta growth factor (PIGF), VEGF-B, VEGF-C, VEGF-D and VEGF-E. Ferrara and Davis-Smyth (1997) Endocr. Rev., supra; Ogawa et al. (1998) J. Biological Chem. 273:31273-31281; Meyer et al. (1999) EMBO J., 18:363-374. A receptor tyrosine kinase, Flt-4 (VEGFR-3), has been identified as the receptor for VEGF-C and VEGF-D. Joukov et al. (1996) EMBO. J. 15:1751; Lee et al. (1996) Proc. Natl. Acad. Sci. USA 93:1988-1992; Achen et al. (1998) Proc. Natl. Acad. Sci. USA 95:548-553. VEGF-C has recently been shown to be involved in the regulation of lymphatic angiogenesis. Jeltsch et al. (1997) Science 276:1423-1425.

Two VEGF receptors have been identified, Flt-1 (also called VEGFR-1) and KDR (also called VEGFR-2). Shibuya et al. (1990) Oncogene 8:519-527; de Vries et al. (1992) Science 255:989-991; Terman et al. (1992) Biochem. Biophys. Res. Commun. 187:1579-1586. Both Flt-I and KDR belong to the family of receptor tyrosine kinases (RTKs). The RTKs comprise a large family of transmembrane receptors with diverse biological activities. At present, at least nineteen (19) distinct RTK subfamilies have been identified. The receptor tyrosine kinase (RTK)-family includes receptors that are crucial for the growth and differentiation of a variety of cell types (Yarden and Ullrich, Ann. Rev. Biochem. 57:433-478, 1988; Ullrich and Schlessinger, Cell 61:243-254, 1990). The intrinsic function of RTKs is activated upon ligand binding, which results in phosphorylation of the receptor and multiple cellular substrates, and subsequently in a variety of cellular responses (Ullrich & Schlessinger, 1990, Cell 61:203-212). Thus, receptor tyrosine kinase mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response. (e.g., cell division, differentiation, metabolic effects, changes in the extracellular microenvironment) see, Schlessinger and Ullrich, 1992, Neuron 9:1-20. Structurally, both Flt-1 and KDR have seven immunoglobulin-like domains in the extracellular domain, a single transmembrane region, and a consensus tyrosine kinase sequence which is interrupted by a kinase-insert domain. Matthews et al. (1991) Proc. Natl. Acad. Sci. USA 88:9026-9030; Terman et al. (1991) Oncogene 6:1677-1683.

There are compelling evidences suggesting that Flt-1 and KDR have different signal transduction properties and possibly mediate different functions. Moreover, the signals mediated through Flt-1 and KDR appear to be cell type specific. Recent studies have provided considerable experimental data linking KDR activation to endothelial cell mitogenesis and chemotaxis in response to VEGF. On the other hand, despite Flt-1's higher binding affinity to VEGF, its function in the adult angiogenesis or other signaling pathways is less understood. Some has suggested that Flt-1 is not primarily a signaling receptor but rather a “decoy” receptor that acts as a negative regulator of the VEGF activity on the vascular endothelium, by sequestering and rendering VEGF less available to the KDR receptor and its own signaling pathway. Park et al. (1994) J. Biol. Chem. 269:25646-54; Hiratsuka et al. (1998) PNAS USA 4:9349-54; U.S. Pat. No. 6,107,046 (Alitalo et al.). Recent studies indicated, however, that Flt-1 has important functions in the VEGF-induced regulation of gene expressions and functions that are specific and important in specific tissues/cells, and that Flt-1 selective VEGF variants can promote proliferation and regeneration of specific tissues such as liver. LeCouter et al. (2003) Science 299:890-893.

The present invention contemplates using VEGF for promoting bone formation and bone repair. In one aspect, VEGF agonists capable of exerting or activating VEGF activity in bone can also be used for the purpose of the invention. Non-limiting examples of agents capable of exerting or activating VEGF activities include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Preferably, VEGF agonists for uses in the present invention are VEGF variants having modified VEGF activities.

The term “VEGF” as used herein refers to the 165-amino acid vascular endothelial cell growth factor and related 121-, 189-, and 206-amino acid vascular endothelial cell growth factors, as described by Leung et al. Science, 246:1306 (1989), and Houck et al. Mol. Endocrin., 5:1806 (1991), together with the naturally occurring allelic and processed forms thereof. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra.).

VEGF and variants thereof for use in the present invention can be prepared by a variety of methods well known in the art. Preferably, the VEGF employed in the methods of the present invention comprises recombinant VEGF₁₆₅. Amino acid sequence variants of VEGF can be prepared by mutations in the VEGF DNA. Such variants include, for example, deletions from, insertions into or substitutions of residues within the amino acid sequence shown in Leung et al., supra and Houck et al., supra. Any combination of deletion, insertion, and substitution may be made to arrive at the final construct having the desired activity. Obviously, the mutations that will be made in the DNA encoding the variant must not place the sequence out of reading frame and preferably will not create complementary regions that could produce secondary mRNA structure. EP 75,444A.

The VEGF variants optionally are prepared by site-directed mutagenesis of nucleotides in the DNA encoding the native VEGF or phage display techniques, thereby producing DNA encoding the variant, and thereafter expressing the DNA in recombinant cell culture.

While the site for introducing an amino acid sequence variation is predetermined, the mutation per se need not be predetermined. For example, to optimize the performance of a mutation at a given site, random mutagenesis may be conducted at the target codon or region and the expressed VEGF variants screened for the optimal combination of desired activity. Techniques for making substitution mutations at predetermined sites in DNA having a known sequence are well-known, such as, for example, site-specific mutagenesis.

Preparation of the VEGF variants described herein is preferably achieved by phage display techniques, such as those described in the PCT publication WO 00/63380.

After such a clone is selected, the mutated protein region may be removed and placed in an appropriate vector for protein production, generally an expression vector of the type that may be employed for transformation of an appropriate host.

Amino acid sequence deletions generally range from about 1 to 30 residues, more preferably 1 to 10 residues, and typically are contiguous.

Amino acid sequence insertions include amino- and/or carboxyl-terminal fusions of from one residue to polypeptides of essentially unrestricted length as well as intrasequence insertions of single or multiple amino acid residues. Intrasequence insertions (i.e., insertions within the native VEGF sequence) may range generally from about 1 to 10 residues, more preferably 1 to 5. An example of a terminal insertion includes a fusion of a signal sequence, whether heterologous or homologous to the host cell, to the N-terminus to facilitate the secretion from recombinant hosts.

Additional VEGF variants are those in which at least one amino acid residue in the native VEGF has been removed and a different residue inserted in its place. Such substitutions may be made in accordance with those shown in Table 1.

TABLE 1

| Original Residue | Exemplary Substitutions |
|---|---|
| Ala (A) | gly; ser |
| Arg (R) | lys |
| Asn (N) | gln; his |
| Asp (D) | glu |
| Cys (C) | ser |
| Gln (Q) | asn |
| Glu (E) | asp |
| Gly (G) | ala; pro |
| His (H) | asn; gln |
| Ile (I) | leu; val |
| Leu (L) | ile; val |
| Lys (K) | arg; gln; glu |
| Met (M) | leu; tyr; ile |
| Phe (F) | met; leu; tyr |
| Ser (S) | thr |
| Thr (T) | ser |
| Trp (W) | tyr |
| Tyr (Y) | trp; phe |
| Val (V) | ile; leu |

Changes in function or immunological identity may be made by selecting substitutions that are less conservative than those in Table 1, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to produce the greatest changes in the VEGF variant properties will be those in which (a) glycine and/or proline (P) is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; (e) a residue having an electronegative side chain is substituted for (or by) a residue having an electropositive charge; or (f) a residue having a bulky side chain, e.g., phenylalanine, is substituted for (or by) one not having such a side chain, e.g., glycine.

The effect of the substitution, deletion, or insertion may be evaluated readily by one skilled in the art using routine screening assays. For example, a phage display-selected VEGF variant may be expressed in recombinant cell culture, and, optionally, purified from the cell culture. The VEGF variant may then be evaluated for KDR or Flt-1 receptor binding affinity and other biological activities, such as those disclosed in the present application. The binding properties or activities of the cell lysate or purified VEGF variant can be screened in a suitable screening assay for a desirable characteristic. For example, a change in the immunological character of the VEGF variant as compared to native VEGF, such as affinity for a given antibody, may be desirable. Such a change may be measured by a competitive-type immunoassay, which can be conducted in accordance with techniques known in the art. The respective receptor binding affinity of the VEGF variant may be determined by ELISA, RIA, and/or BIAcore assays, known in the art and described further in the Examples below. Preferred VEGF variants of the invention will also exhibit activity in KIRA assays (such as described in the Examples) reflective of the capability to induce phosphorylation of the KDR receptor. Preferred VEGF variants of the invention will additionally or alternatively induce endothelial cell proliferation (which can be determined by known art methods such as the HUVEC proliferation assay in the Examples). In addition to the specific VEGF variants disclosed herein, the VEGF variants described in Keyt et al. J. Biol. Chem., 271:5638-5646 (1996) are also contemplated for use in the present invention.

VEGF or variants thereof and methods of making the same have been known and described in, for example, the PCT publication WO 00/63380 and Li et al. (2000) J. Biol. Chem. 275:29823-29828. In some aspects, VEGF variants with one or more amino acid mutations are Flt-1 selective, exhibiting binding affinity to the Flt-1 receptor which is equal to or greater (≧) than the binding affinity of native VEGF to the Flt-1 receptor, and even more preferably, such VEGF variants exhibit less binding affinity (<) to the KDR receptor than the binding affinity exhibited by native VEGF to KDR. When binding affinity of such VEGF variant to the Flt-1 receptor is approximately equal (unchanged) or greater than (increased) as compared to native VEGF, and the binding affinity of the VEGF variant to the KDR receptor is less than or nearly eliminated as compared to native VEGF, the binding affinity of the VEGF variant, for purposes herein, is considered “selective” for the Flt-1 receptor. Preferred Flt-1 selective VEGF variants of the invention will have at least 10-fold less binding affinity to KDR receptor (as compared to native VEGF), and even more preferably, will have at least 100-fold less binding affinity to KDR receptor (as compared to native VEGF). The respective binding affinity of the VEGF variant may be determined by ELISA, RIA, and/or BIAcore assays, known in the art and described in the PCT publication WO 00/63380.

In some other aspects of the invention, the VEGF variants are KDR selective, capable of selectively binding to KDR (referred hereinafter as “KDR selective VEGF”). KDR selective VEGF variants and methods of making the same are described in detail in the Example sections below. Additional disclosures relating to KDR selective VEGF can be found in, for example, the PCT publication WO 00/63380 and Li et al. (2000) J. Biol. Chem. 275:29823-29828. Preferred KDR selective VEGF variants include one or more amino acid mutations and exhibit binding affinity to the KDR receptor which is equal to or greater (≧) than the binding affinity of native VEGF to the KDR receptor, and even more preferably, the VEGF variants exhibit less binding affinity (<) to the flt-1 receptor than the binding affinity exhibited by native VEGF to flt-1. When binding affinity of such VEGF variant to the KDR receptor is approximately equal (unchanged) or greater than (increased) as compared to native VEGF, and the binding affinity of the VEGF variant to the fit-1 receptor is less than or nearly eliminated as compared to native VEGF, the binding affinity of the VEGF variant, for purposes herein, is considered “selective” for the KDR receptor. Preferred KDR selective VEGF variants of the invention will have at least 10-fold less binding affinity to Flt-1 receptor (as compared to native VEGF), and even more preferably, will have at least 100-fold less binding affinity to Flt-1 receptor (as compared to native VEGF). The respective binding affinity of the VEGF variant may be determined by ELISA, RIA, and/or BIAcore assays that are known in the art. Preferred KDR selective VEGF variants of the invention will also exhibit activity in KIRA assays reflective of the capability to induce phosphorylation of the KDR receptor. Preferred KDR selective VEGF variants of the invention will additionally or alternatively induce endothelial cell proliferation (which can be determined by known methods such as the HUVEC proliferation assay).

In one aspect, VEGF and variants thereof for use in the present invention are produced by recombinant methods. Isolated DNA used in these methods is understood herein to mean chemically synthesized DNA, cDNA, chromosomal, or extrachromosomal DNA with or without the 3′- and/or 5′-flanking regions. Preferably, the VEGF and variants thereof herein are made by synthesis in recombinant cell culture.

For such synthesis, it is first necessary to secure nucleic acid that encodes a VEGF or VEGF variant. DNA encoding a VEGF molecule may be obtained from bovine pituitary follicular cells by (a) preparing a cDNA library from these cells, (b) conducting hybridization analysis with labeled DNA encoding the VEGF or fragments thereof (up to or more than 100 base pairs in length) to detect clones in the library containing homologous sequences, and (c) analyzing the clones by restriction enzyme analysis and nucleic acid sequencing to identify full-length clones. If full-length clones are not present in a cDNA library, then appropriate fragments may be recovered from the various clones using the nucleic acid sequence information disclosed herein for the first time and ligated at restriction sites common to the clones to assemble a full-length clone encoding the VEGF. Alternatively, genomic libraries will provide the desired DNA.

Once this DNA has been identified and isolated from the library, it is ligated into a replicable vector for further cloning or for expression.

In one example of a recombinant expression system, a VEGF-encoding gene is expressed in a cell system by transformation with an expression vector comprising DNA encoding the VEGF. It is preferable to transform host cells capable of accomplishing such processing so as to obtain the VEGF in the culture medium or periplasm of the host cell, i.e., obtain a secreted molecule.

“Transfection” refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

“Transformation” refers to introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described by Cohen, Proc. Natl. Acad. Sci. (USA), 69: 2110 (1972) and Mandel et al J. Mol. Biol., 53: 154 (1970), is generally used for prokaryotes or other cells that contain substantial cell-wall barriers. For mammalian cells without such cell walls, the calcium phosphate precipitation method of Graham and van der Eb, Virology, 52: 456-457 (1978), is preferred. General aspects of mammalian cell host system transformations have been described by Axel in U.S. Pat. No. 4,399,216 issued Aug. 16, 1983. Transformations into yeast are typically carried out according to the method of Van Solingen et al. J. Bact., 130. 946 (1977) and Hsiao et al. Proc. Natl. Acad. Sci. (USA), 76. 3829 (1979). However, other methods for introducing DNA into cells such as by nuclear injection or by protoplast fusion may also be used.

The vectors and methods disclosed herein are suitable for use in host cells over a wide range of prokaryotic and eukaryotic organisms.

In general, of course, prokaryotes are preferred for the initial cloning of DNA sequences and construction of the vectors useful in the invention. For example, E. coli K 12 strain MM 294 (ATCC No. 31,446) is particularly useful. Other microbial strains that may be used include E. coli strains such as E. coli B and E. coli X1776 (ATCC No. 31,537). These examples are, of course, intended to be illustrative rather than limiting.

Prokaryotes may also be used for expression. The aforementioned strains, as well as E. coli strains W3110 (F-, lambda-, prototrophic, ATCC No. 27,325), K5772 (ATCC No. 53,635), and SR101, bacilli such as Bacillus subtilis, and other enterobacteriaceae such as Salmonella typhimurium or Serratia marcesans, and various pseudomonas species, may be used.

In general, plasmid vectors containing replicon and control sequences that are derived from species compatible with the host cell are used in connection with these hosts. The vector ordinarily carries a replication site as well as marking sequences that are capable of providing phenotypic selection in transformed cells. For example, E. coli is typically transformed using pBR322, a plasmid derived from an E. coli species (see, e.g., Bolivar et al. Gene, 2:95 (1977). pBR322 contains genes for ampicillin and tetracycline resistance and thus provides easy means for identifying transformed cells. The pBR322 plasmid, or other microbial plasmid or phage, must also contain, or be modified to contain, promoters that can be used by the microbial organism for expression of its own proteins.

Those promoters most commonly used in recombinant DNA construction include the β-lactamase (penicillinase) and lactose promoter systems Chang el al. Nature, 375:615 (1978); Itakura et al. Science, 198:1056 (1977); Goeddel et al. Nature, 281:544 (1979)) and a tryptophan (trp) promoter system (Goeddel et al. Nucleic Acids Res., 8:4057 (1980); EPO Appl. Publ. No. 0036,776). While these are the most commonly used, other microbial promoters have been discovered and utilized, and details concerning their nucleotide sequences have been published, enabling a skilled worker to ligate them functionally with plasmid vectors (see, e.g., Siebenlist et al. Cell, 20:269 (1980)).

In addition to prokaryotes, eukaryotic microbes, such as yeast cultures, may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al. Nature, 282:39 (1979); Kingsman et al. Gene, 7:141 (1979); Tschemper et al. Gene, 10:157 (1980)), is commonly used. This plasmid already contains the trp1 gene that provides a selection marker for a mutant strain of yeast lacking the ability to grow in tryptophan, for example, ATCC No. 44,076 or PEP4-1 (Jones, Genetics, 85:12 (1977)). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzeman et al. J. Biol. Chem., 255:2073 (1980)) or other glycolytic enzymes (Hess et al. J. Adv. Enzyme Reg., 7:149 (1968); Holland et al. Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination. Other promoters, which have the additional advantage of transcription controlled by growth conditions, are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years (Tissue Culture, Academic Press, Kruse and Patterson, editors (1973)). Examples of such useful host cell lines are VERO and HeLa cells, Chinese hamster ovary (CHO) cell lines, and W138, BHK, COS-7, 293, and MDCK cell lines. Expression vectors for such cells ordinarily include (if necessary) an origin of replication, a promoter located in front of the gene to be expressed, along with any necessary ribosome binding sites, RNA splice sites, polyadenylation sites, and transcriptional terminator sequences.

For use in mammalian cells, the control functions on the expression vectors are often provided by viral material. For example, commonly used promoters are derived from polyoma, Adenovirus2, and most frequently Simian Virus 40 (SV40). The early and late promoters of SV40 virus are particularly useful because both are obtained easily from the virus as a fragment that also contains the SV40 viral origin of replication (Fiers et al. Nature, 273:113 (1978)). Smaller or larger SV40 fragments may also be used, provided there is included the approximately 250-bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

Satisfactory amounts of protein are produced by cell cultures; however, refinements, using a secondary coding sequence, serve to enhance production levels even further. One secondary coding sequence comprises dihydrofolate reductase (DHFR) that is affected by an externally controlled parameter, such as methotrexate (MTX), thus permitting control of expression by control of the methotrexate concentration.

In selecting a preferred host cell for transfection by the vectors of the invention that comprise DNA sequences encoding both VEGF and DHFR protein, it is appropriate to select the host according to the type of DHFR protein employed. If wild-type DHFR protein is employed, it is preferable to select a host cell that is deficient in DHFR, thus permitting the use of the DHFR coding sequence as a marker for successful transfection in selective medium that lacks hypoxanthine, glycine, and thymidine. An appropriate host cell in this case is the Chinese hamster ovary (CHO) cell line deficient in DHFR activity, prepared and propagated as described by Urlaub and Chasin, Proc. Natl. Acad. Sci. (USA), 77:4216 (1980).

On the other hand, if DHFR protein with low binding affinity for MTX is used as the controlling sequence, it is not necessary to use DHFR-deficient cells. Because the mutant DHFR is resistant to methotrexate, MTX-containing media can be used as a means of selection provided that the host cells are themselves methotrexate sensitive. Most eukaryotic cells that are capable of absorbing MTX appear to be methotrexate sensitive. One such useful cell line is a CHO line, CHO-K1 (ATCC No. CCL 61).

Construction of suitable vectors containing the desired coding and control sequences employs standard ligation techniques. Isolated plasmids or DNA fragments are cleaved, tailored, and religated in the form desired to prepare the plasmids required.

If blunt ends are required, the preparation may be treated for 15 minutes at 15° C. with 10 units of Polymerase I (Klenow), phenol-chloroform extracted, and ethanol precipitated.

Size separation of the cleaved fragments may be performed using, by way of example, 6 percent polyacrylamide gel described by Goeddel et al. Nucleic Acids Res., 8:4057 (1980).

To confirm correct sequences were constructed in plasmids, the ligation mixtures are typically used to transform E. coli K12 strain 294 (ATCC 31,446) or other suitable E. coli strains, and successful transformants selected by ampicillin or tetracycline resistance where appropriate. Plasmids from the transformants are prepared and analyzed by restriction mapping and/or DNA sequencing by the method of Messing et al. Nucleic Acids Res., 9:309 (1981) or by the method of Maxam et al. Methods of Enzymology, 65:499 (1980).

After introduction of the DNA into the mammalian cell host and selection in medium for stable transfectants, amplification of DHFR-protein-coding sequences is effected by growing host cell cultures in the presence of approximately 20,000-500,000 nM concentrations of methotrexate (MTX), a competitive inhibitor of DHFR activity. The effective range of concentration is highly dependent, of course, upon the nature of the DHFR gene and the characteristics of the host. Clearly, generally defined upper and lower limits cannot be ascertained. Suitable concentrations of other folic acid analogs or other compounds that inhibit DHFR could also be used. MTX itself is, however, convenient, readily available, and effective.

Compounds useful in the invention include small organic molecules that exert their activating functions at the intracellular tyrosine kinase domain of the RTKs. In certain preferred embodiments, small molecule VEGF agonists are employed to stimulate one or both VEGF receptors, thereby activating the corresponding signaling pathway. Many small to molecule compounds can be used for the purpose of this invention. These include, but not limited to, bis monocyclic, bicyclic or heterocyclic aryl compounds, vinylene-azaindole derivatives (PCT WO 94/14808) and 1-cycloproppyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), selenoindoles and selenides (PCT WO 94/03427), tricyclic polyhydroxylic compounds (PCT WO 92/21660) and benzylphosphonic acid compounds (PCT WO 91/15495).

Treatment of Bone Defects

According to one embodiment, the invention provides methods for treating a pathological bone defect in a subject. As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, improving the rate of recovery, amelioration or palliation of the disease state, and remission or improved prognosis.

An “effective amount” refers to an amount effective, at dosages and for periods of time necessary, to achieve the desired therapeutic or prophylactic result. A “therapeutically effective amount” of an agent may vary according to factors such as the disease state, age, sex, and weight of the individual, and the ability of the agent to elicit a desired response in the individual. A therapeutically effective amount is also one in which any toxic or detrimental effects of the agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The phrase “bone defect” as used herein refers to any structural and/or functional skeletal abnormalities. Non-limiting examples of bone defect include those associated with vertebral body or disc injury/destruction, spinal fusion, injured meniscus, avascular necrosis, cranio-facial repair/reconstruction, cartilage destruction/damage, osteoarthritis, osteosclerosis, osteoporosis, implant fixation, and other inheritable or acquired bone disorders and diseases. One of the most common bone defect is fracture associated with a trauma (caused by, for example, physical injury) or with degenerative disorders.

Pharmaceutical Compositions and Therapeutic/prophylactic Administration

For in vivo uses according to the methods of the invention, a therapeutic compound of the invention is administered to a subject using methods and techniques known in the art and suitable for the particular use. In a preferred embodiment, the compound is administered in the form of pharmaceutical compositions at a pharmaceutically acceptable dosage. Such compositions may comprise a therapeutically effective amount of VEGF or variant thereof, and a pharmaceutically acceptable carrier. In a specific embodiment, the term “pharmaceutically acceptable” means approved by a regulatory agency of the Federal or a state government or listed in the U.S. Pharmacopoeia or other generally recognized pharmacopoeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

Preferably, the therapeutic agent of the invention is administered by a delivery system that provides local, sustained therapeutic activity of the agent. One preferred system uses a slow-release preparation of the therapeutic agent. Suitable examples of slow-release preparations include semipermeable matrices of solid hydrophobic polymers containing the multivalent antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

As an specific example, PLAD, a bio-erodible polyactic acid depot, is used for the slow release of VEGF or variant thereof at the site of bone fracture according to the present invention. PLAD combines polyactic acid with hydrophilic (benzyl alcohol) and hydrophobic (benzyl benzoate) solvents to dissolve protein and solvate PLA, respectively. PLAD is described in further detail in the following Examples and in Cleland et al (2001) J. Contr. Rel. 72:13-24, the disclosure of which is incorporated herein by reference.

It is further contemplated that a therapeutic protein agent of the invention (such as VEGF or VEGF variant) can be introduced to a subject by gene therapy. Gene therapy refers to therapy performed by the administration of a nucleic acid to a subject. For general reviews of the methods of gene therapy, see, for example, Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; Morgan and Anderson (1993) Ann. Rev. Biochem. 62:191-217; and May (1993) TIBTECH 11:155-215. Methods commonly known in the art of recombinant DNA technology which can be used are described in Ausubel et al. eds. (1993) Current Protocols in Molecular Biology, John Wiley & Sons, NY; and Kriegler (1990) Gene Transfer and Expression, A Laboratory Manual, Stockton Press, NY.

There are two major approaches to getting the nucleic acid (optionally contained in a vector) into the subject's cells; in vivo and ex vivo. For in vivo delivery the nucleic acid is injected directly into the subject, usually at the site where the protein is required. For ex vivo treatment, the subject's cells are removed, the nucleic acid is introduced into these isolated cells and the modified cells are administered to the subject either directly or, for example, encapsulated within porous membranes which are implanted into the subject (see, e.g. U.S. Pat. Nos. 4,892,538 and 5,283,187). There are a variety of techniques available for introducing nucleic acids into viable cells. The techniques vary depending upon whether the nucleic acid is transferred into cultured cells ex vivo, or in vivo in the cells of the intended host. Techniques suitable for the transfer of nucleic acid into mammalian cells ex vivo include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, lentivirus, retrovirus, or adeno-associated virus) and lipid-based systems (useful lipids for lipid-mediated transfer of the gene are DOTMA, DOPE and DC-Chol, for example). Examples of using viral vectors in gene therapy can be found in Clowes et al. (1994) J. Clin. Invest. 93:644-651; Kiem et al. (1994) Blood 83:1467-1473; Salmons and Gunzberg (1993) Human Gene Therapy 4:129-141; Grossman and Wilson (1993) Curr. Opin. in Genetics and Devel. 3:110-114; Bout et al. (1994) Human Gene Therapy 5:3-10; Rosenfeld et al. (1991) Science 252:431-434; Rosenfeld et al. (1992) Cell 68:143-155; Mastrangeli et al. (1993) J. Clin. Invest. 91:225-234; and Walsh et al. (1993) Proc. Soc. Exp. Biol. Med. 204:289-300.

In some situations it is desirable to provide the nucleic acid source with an agent that targets the target cells, such as an antibody specific for a cell surface membrane protein on the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al. J. Biol. Chem. 262:4429-4432 (1987); and Wagner et al. Proc. Natl. Acad. Sci. USA 87:3410-3414 (1990). For review of the known gene marking and gene therapy protocols see Anderson et al. Science 256:808-813 (1992).

The therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc. The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Articles of Manufacture

In another embodiment of the invention, an article of manufacture containing materials useful for the treatment of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a multivalent antibody. The label or package insert indicates that the composition is used for treating the condition of choice, such as cancer. Moreover, the article of manufacture may comprise (a) a first container with a composition contained therein, wherein the composition comprises a multivalent antibody; and (b) a second container with a composition contained therein, wherein the composition comprises a further cytotoxic agent. The article of manufacture in this embodiment of the invention may further comprises a package insert indicating that the first and second antibody compositions can be used to treat cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The invention will be more fully understood by reference to the following examples. They should not, however, be construed as limiting the scope of this invention. All literature and patent citations mentioned herein are expressly incorporated by reference.

### EXAMPLES

### Example 1

### Endogenous VEGF is Required for Normal Bone Repair

Murine Models of Bone Defects and Repair

To create a model for endochondral ossification, i.e., femoral fracture healing, 6-8 weeks old male C57 B16 mice were anaesthetized using Avertin (12.8 g/L Tribromoethanol, 10 ml/kg body weight). Under asepsis and following sterile preparation of a midline incision in the skin overlying the right knee joint was performed and a 22″ hypodermic needle was inserted in a retrograde fashion through the quadriceps tendon, through the intercondylar area and into the shaft of the right femur. The “pin” was advanced until it engaged the greater trochanter proximally. The mice (n=175) were placed on a custom-made jig and a weight was dropped (from 25 cm) impacting the midshaft of the pinned right femur. Bonnarens and Einhorn (1984) J. Orthop. Res. 2:97-101. Following creation of the bony injury, the fracture site was opened through a lateral skin incision and a muscle splitting lateral approach. In the first group, the periosteum, a region critical for healing, was left intact; in the group with a “challenged” fracture, the periosteum was circumferentially stripped for 2.0 mm proximal and distal to the fracture.

Radiographs (Faxitron X-ray, MX20, IDL software), taken at the time of fracture and weekly thereafter, were used to confirm the position of fractures and pins. Any animals in which the pin came out, the fracture was grossly displaced, or the fracture was not midshaft were not analyzed. Because only the mice which satisfied these inclusion criteria were used, the sample size in each group was unequal, but each group had a minimum of 7 animals.

Mice were either untreated (control) or given intraperitoneal injections (25 mg/kg) of a control antibody (anti-glycoprotein D) or the murine Flt-1 IgG (Ferrara et al (1998) Nat. Med. 4:336-340) on alternate days until euthanasia.

To create a murine model of intramembranous bone repair, i.e., a focal cortical defect in the tibia, a full thickness unicortical defect was created with a dental burr (1 mm) on the anteriomedial aspect of the right tibia of the mice. Continuous saline irrigation was employed during burring to prevent thermal necrosis of margins. The same mice also underwent femoral fractures as described above. The mice were returned to their cages and allowed to mobilize freely.

Computed Tomography (CT) Analysis

X-ray micro-computed tomography (ACT) images were acquired at 50 keV and 80 microamperes (μA) (mice) or 160 μA (rabbits) using a SCANCO Medical (Bassersdorf, Switzerland) μCT20/40. Axial images were obtained with an in-plane resolution of 26×26 μm, slice thickness of 35 μm, and an inter-slice gap of 69 μm (mice) or contiguous slices with voxel dimensions of 30×30×31 μm (rabbits). A hydroxyapatite (HA) phantom of known density (2.91 g/cm³) was used for system calibration. Callus volume and mean voxel intensity were calculated for each model using a callus volume of interest (VOI_{callus}) A “calcification” threshold was applied to VOI_{callus} to determine volume and mean intensity of highly calcified callus. The calcified callus threshold (0.48 gHA/cm³) was set at 50% of the minimum intensity found to segment cortical bone. Percent calcified callus was defined as the ratio of calcified callus volume to total callus volume. VOI_{callus} for mouse femurs and tibiae was determined by manual segmentation with the SCANCO image analysis software package. VOI_{callus} (rabbit) was determined by application of an automated image segmentation algorithm (Analyze software, AnalyzeDirect Inc., Lenexa, Kans.). Lower and upper thresholds (0.22 and 1.33 gHA/cm³, respectively), determined by histogram analysis of data from three rabbits, were applied to extract potential callus voxels. A series of morphological filtering operations (erode, open, conditional dilate, and close) was then applied to extract the callus volumes. The algorithm results were highly correlated (volume: r=0.99, P<0.01; density: r=0.96, P<0.01) with manual estimates of callus parameters for an independent group of eight rabbit bones.

In-Vivo PECAM-1 Labeling

Monoclonal rat anti-mouse antibody PECAM-1 IgG₂ₐ (Pharmingen Inc., San Diego, Calif., clone MEC13.3) labeled with 125I (Dupont NEN, Boston, Mass., NEZ-033A) and a non-specific isotype control antibody, rat anti-mouse CD 35, IgG₂ₐ (Pharmingen Inc., San Diego, Calif., clone 8C12) labeled with ¹³¹I (Dupont NEN, Boston, Mass., NEZ-035A) were used according to Vecchi et al. (1994) Eur. J. Cell. Biol. 63:247-54; Eppihimer et al (1998) Microcirc. 5:179-188; and Panes et al. (1995) Am. J. Physiol. 269:H1955-64. All antibodies were iodinated using the iodogen method in a ratio of 1 μg of antibody to 1 μCi of either ¹²⁵I or ¹³¹I. To measure PECAM-1 binding, a mixture of ¹²⁵I PECAM-1 mAb (10 μg) and ¹³¹I non-specific mAb (equivalent to 500,000 cpm) was diluted with PBS (to 200 μl). Starting radioactivity (2 μl) was counted (Wallac Wizard 3″ gamma counter, model 1480, PerkinElmer, Gaithersburg, Md.). Cold PECAM-1 mAb (30 μg) was added, and the mixture was injected through the jugular vein catheter and allowed to circulate for 5 minutes. Blood samples were obtained from carotid catheters to measure circulating radiolabeled antibody levels. Animals were exsanguinated by perfusion with 6 mls bicarbonate buffered saline (BBS) through the jugular catheter with simultaneous blood withdrawal from the carotid catheter. This was followed by perfusion of BBS through the carotid catheter (15 mls) after severing the inferior vena cava at the thoracic level. Organs and muscles were collected, weighed, and radioactivity was counted.

Endogenous VEGF is Essential for Normal Fracture Repair (Endochondral Ossification)

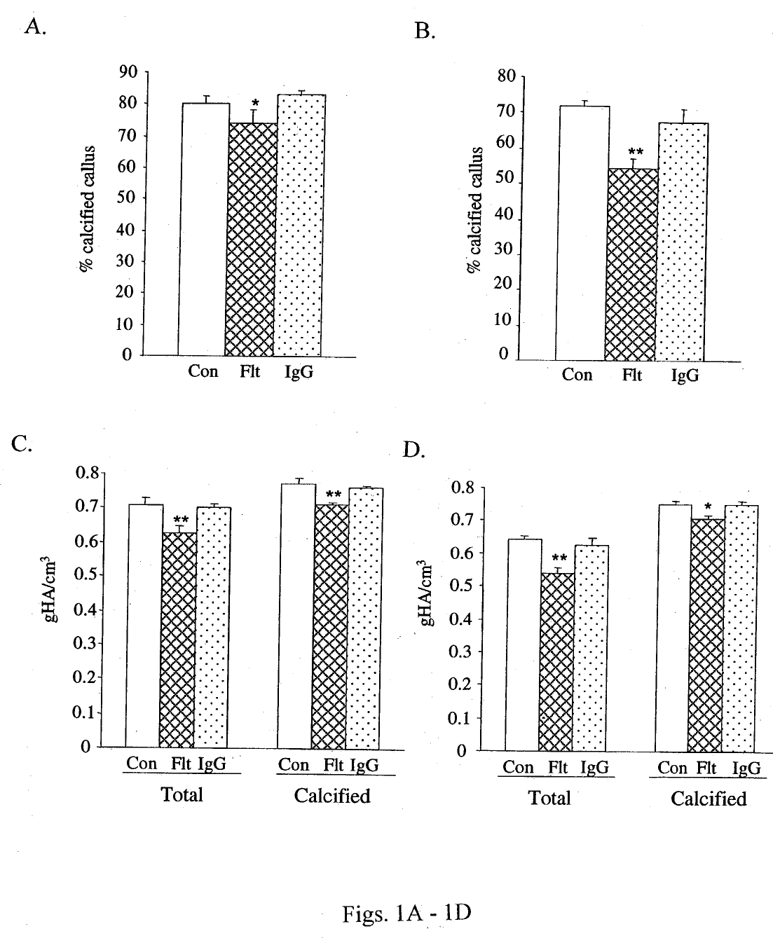

Treatment of mice with the soluble VEGF receptor Flt-IgG, a VEGF antagonist, during the course of fracture repair, dramatically impaired normal healing (via endochondral ossification) as illustrated in three-dimensional (3D) renderings of mouse femur fractures. Repair was examined at post-fracture time points corresponding to soft (cartilaginous) callus (7 days) and to hard (bony) callus (14 days). Using quantitative computed tomography (CT), The results showed that that volumes of the total callus (8.15±1.05 mm³, Control; 8.30±0.95 mm³, control IgG; 5.35±0.49 mm³, Flt-IgG) and calcified callus (6.41±0.79 mm³, Control; 6.91±0.83 mm³, control IgG; vs. 3.88±0.24 mm³, Flt-IgG) were decreased by 35.5% (P=0.03) and 43.9% (P=0.01), respectively in Flt-IgG treated mice at 7 days, but not at 14 days after fracture relative to mice treated with control antibodies. However, percent callus calcified was decreased at both timepoints, i.e. by 10.7% (P=0.04) at 7 days and by 19.4% (P=0.008) at 14 days in Flt-IgG treated mice relative to animals treated with control antibodies (FIGS. 1a, 1b). Similarly, mineral density was decreased by 10.7% in the total callus (P=0.005) and 6.4% in the calcified callus (P=0.0006) at 7 days, and 13.4% in the total callus (P=0.005) and 5.7% in the calcified callus (P=0.02) at 14 days in Flt-IgG treated mice relative to animals treated with control antibodies (FIGS. 1c, 1d). Histologic examination of the fracture callus at 7 days post-fracture showed wider callus trabeculae and persistent perinuclear osteocyte lacunae in the fracture callus of Flt-IgG-treated mice relative to both sets of control mice, suggesting decreased callus maturity in Flt-IgG treated mice.

Endogenous VEGF is Required for Normal Repair of Focal Cortical Bone Defects (Intramembranous Ossification)

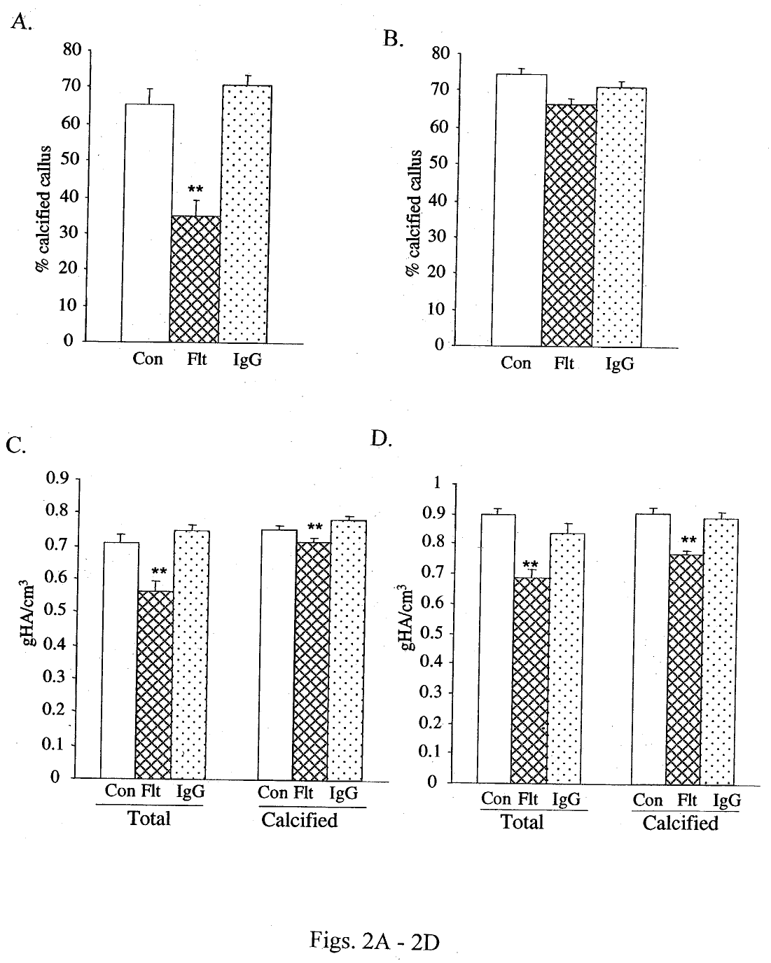

Unlike unstable femur fractures which heal through endochondral ossification, rigid, cortical bone heals through intramembranous (direct) ossification. CT analysis indicated that healing of a focal defect in the proximal tibial cortex was impaired in Fit-IgG treated mice. Relative to control antibody (IgG) treated animals, calcified callus volume was reduced by 60.7% (0.247±0.016 mm³, Control; 0.277=0.019 mm³, control IgG; 0.109±0.012 mm³, Flt-IgG; P=4.5×10⁻⁷), and percent calcification was 50.7% lower (P=3.04×10-7) at 7 days (FIG. 2a), but only trended lower (P=0.095) at 14 days (FIG. 2b), in Flt-IgG treated mice relative to IgG treated mice. Treatment with Flt-IgG decreased 7 day total callus mean mineral density by 24.7% (P=1.11×10⁻⁵) and calcified mean mineral density by 7.9% (P=0.004) (FIG. 2c) and 14 day total callus mean mineral density by 17.6% (P=0.003) and calcified callus mean mineral density by 14% (P=0.0003) (FIG. 2d). In contrast to the well-mineralized, woven bone in defect sites in control animals at 7 days after surgery, Flt-IgG-treated animals showed largely unresorbed, uncalcified hematoma in the medullary space. At 14 days after surgery, mineralized bone was present at the defect site of all mice, but control animals showed evidence of remodelling to lamellar bone, whereas in Flt-IgG animals, defects were still largely composed of woven bone with less well-organized collagen fibrils when viewed in polarized light.

Endogenous VEGF Promotes Callus Vascularity

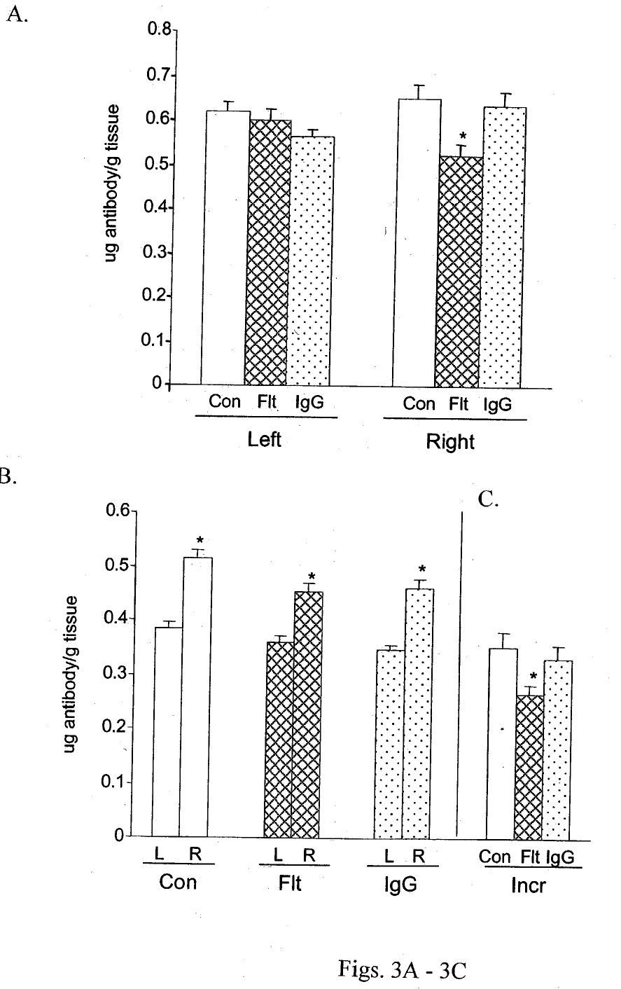

Vascularity, as quantitated by platelet-endothelial cell adhesion molecule (PECAM) expression was reduced by 18% (P=0.01) in fractured bones of Flt-IgG treated animals relative to control or IgG-treated mice at 7 days after fracture (FIG. 3a). Due to injury, PECAM expression in soft tissues of fractured (right) legs was increased relative to contra-lateral, intact (left) legs in all groups: Control (P=0.00006), IgG treated (P=0.00003), and Flt-IgG treated mice (P=0.001) (FIG. 3b). However, percent induction of vascularity in the fractured leg relative to the untreated, contra-lateral leg was 20.4% less (P=0.036) in Flt-IgG treated mice relative to control antibody (IgG) treated mice and 25% less (P=0.01) in Flt-IgG treated mice relative to untreated control mice (FIG. 3c). No significant difference in vascular induction between Control and IgG-treated mice was found.

Our Flt-IgG studies show that VEGF inhibition in mice resulted in disruption in repair of femoral fractures (endochondral ossification) and cortical bone defects (intramembranous ossification). Our data provide the first evidence that VEGF activity is essential for production of appropriate callus formation and mineralization in response to bone injury.

Fracture repair occurs in a series of steps, involving an initial inflammatory phase, a soft callus (cartilage) phase, a hard callus (bone) phase, and a remodeling phase. The timepoints in our mouse fracture study corresponded to the soft callus phase (day 7) and the hard callus phase (day 14) during normal fracture repair. Consistent with the fact that endogenous VEGF is expressed in hypertrophic chondroctyes at ˜10 days after fracture in mice, Flt-IgG affected calcification of the hard callus 14 days after fracture. Our data show that VEGF is involved in conversion of the soft, cartilaginous callus (7 days) to a hard, bony callus (14 d) during fracture repair just as VEGF couples angiogenesis, cartilage resorption, and ossification in the growth plate of developing mice (Gerber and Ferrara (2000) Trends Cardiovasc. Med. 10:223-228). Our studies of vascularity at 7 days after fracture indicate the importance of VEGF in early angiogenesis in both the fractured bone as well as the surrounding soft tissues. In the early stages of bone repair, the fracture hematoma serves as a reservoir for large amounts of active VEGF, a VEGF source which is not present in developing bones. Thus, Flt-IgG inhibition of soft callus formation at 7 days after fracture could not have been predicted based on developmental studies. Our results show a novel role for VEGF in the early stages of fracture repair and highlight differences in the processes of endochondral ossification during development vs. that in fracture repair.

As in the fracture model, Flt-IgG treatment impaired healing at 7 days in the cortical bone defect model. The persistence of unresorbed, unmineralized fracture hematoma in this defect at 7 days and the persistence of woven bone at 14 days indicate remodelling defects in Flt-IgG treated mice. Such results support the findings that VEGF stimulates chemotaxis of osteoclasts and osteoclast activity. Engsig et al. (2000) J. Cell. Biol. 151:879-889; Niida et al. (1999) J. Exp. Med. 190:293-298. Unlike the timing of Flt-IgG effects in our endochondral bone formation model (fractures), healing defects in Flt-IgG treated mice were more less prominent later in the process of intramembranous bone formation. The difference in the effect of Flt-IgG at later timepoints in the two models is related to the fact that intramembranous ossification (in the cortical defect) does not involve a cartilage intermediate. Thus, the mechanism of Flt-IgG inhibition of repair of the cortical bone defect at 14 days is distinct from that in the fracture model. In the absence of cartilage, osteoblasts are likely producing, and responding to VEGF, in the cortical bone defect as indicated by our in vitro data presented in the following Example, showing hypoxia-induced expression of VEGF, but not FGF, in primary osteoblasts. The decrease in mean mineral density at 14 days in the tibia defect might be explained by interference with stimulation of osteoblasts by VEGF as our in vitro data with anti-VECF antibodies indicate. Thus, in the cortical bone defect, VEGF appears to be involved in recruitment and activation of osteoclasts at early stages of repair, while later in the healing process, VEGF can promote differentiation of osteoblasts and matrix mineralization. Our results are the first to implicate VEGF in intramembranous bone repair.

### Example 2

### VEGF Directly Promotes Osteoblast Differentiation

To further characterize the role of endogenous VEGF in osteoblast activity independent of VEGF's activity on endothelial cells, primary human osteoblasts in vitro were studied. Primary normal human osteoblasts were cultured from trabecular bone explants obtained at the time of orthopaedic procedures performed on consenting young adults who had no evidence of metabolic bone disease. The bone fragments were washed extensively and repeatedly with culture medium to remove adherent marrow cells and to expose the trabecular surface of the bone. Small bone chips (1×1×1 mm) were then placed in culture flasks (75 cm²), each containing 15 mls α-modified Earle's medium (αMEM) supplemented with 10% heat inactivated fetal calf serum, penicillin (100 U/mL), streptomycin (50 μg/mL) and cultured at 37° C. in a humidified atmosphere with 5% CO₂. Cell outgrowth from the trabecular bone surfaces was apparent after 5 days, and osteoblast-like cells became confluent after 10-14 days in culture. Verification of osteoblast lineage was performed by mineralized bone nodule formation assay, alkaline phosphatase activity of the cell lysate using sodium p-nitrophenyl phosphate substrate, and by FACS analysis for osteocalcin (96.8-98.4% cell purity). Cell passages were performed by incubating confluent cells 0.25% trypsin diluted in calcium and magnesium free phosphate buffered saline. Experiments were performed on osteoblasts subcultured to passage 3-6.

To assay for bone nodule formation, primary human osteoblasts were seeded in 6-well plates at a density of 1×10⁵ cells/mL and cultured as above. Upon confluence (48-72 hours after plating), 50 ug/ml ascorbic acid was added to the cultures. The cell cultures were then supplemented with recombinant human VEGF 165 (0-50 ng/mL) or a monoclonal mouse anti-human VEGF neutralizing antibody (0.3 μg/mL). The treated medium was replenished daily. Mineralized nodules began to appear by 3-8 days at which time the medium was further supplemented with beta-glycerol-phosphate and ascorbic acid to further stimulate osteogenic differentiation. After 18 days in culture, the number of mineralized bone nodules was quantified by von Kossa staining. All cultures were performed in triplicate, and six fields per culture well were counted.

Alkaline phosphatase activity in the osteoblast culture system was determined by measuring cell supernatant hydrolysis of p-nitrophenyl phosphate, yielding p-nitrophenol, which when alkaline is converted to a yellow complex easily measured by spectophotometric analysis at 400-420 nm (Sigma Diagnostics).

To assay for VEGF production, primary human osteoblasts were trypsinized and then incubated in 500 μL of culture medium in 24 well plates at 1×10⁵ cells per well for 6-8 hours until cells became adherent. Culture medium was then replenished, and cells were cultured for 6, 12, 18, 24, 36 or 48 hours. At the end of the incubation time, the cell supernatant was carefully removed, centrifuged at 3000 g for 10 minutes at 4° C., passed through a 0.2 μm sterile filter and used immediately for cytokine determination or aliquoted and stored at −80° C. for up to one week prior to analysis. Osteoblast conditioned medium was assayed for the angiogenic cytokines human vascular endothelial growth factor (VEGF) and basic fibroblast growth factor (bFGF) by the quantitative sandwich enzyme-linked immunosorbant assay (ELISA) technique, using Quantikine kits (R&D Systems), according to the manufacturers protocols. The sensitivity for VEGF and bFGF were <5.0 pg/mL and <3.0 pg/mL, respectively.

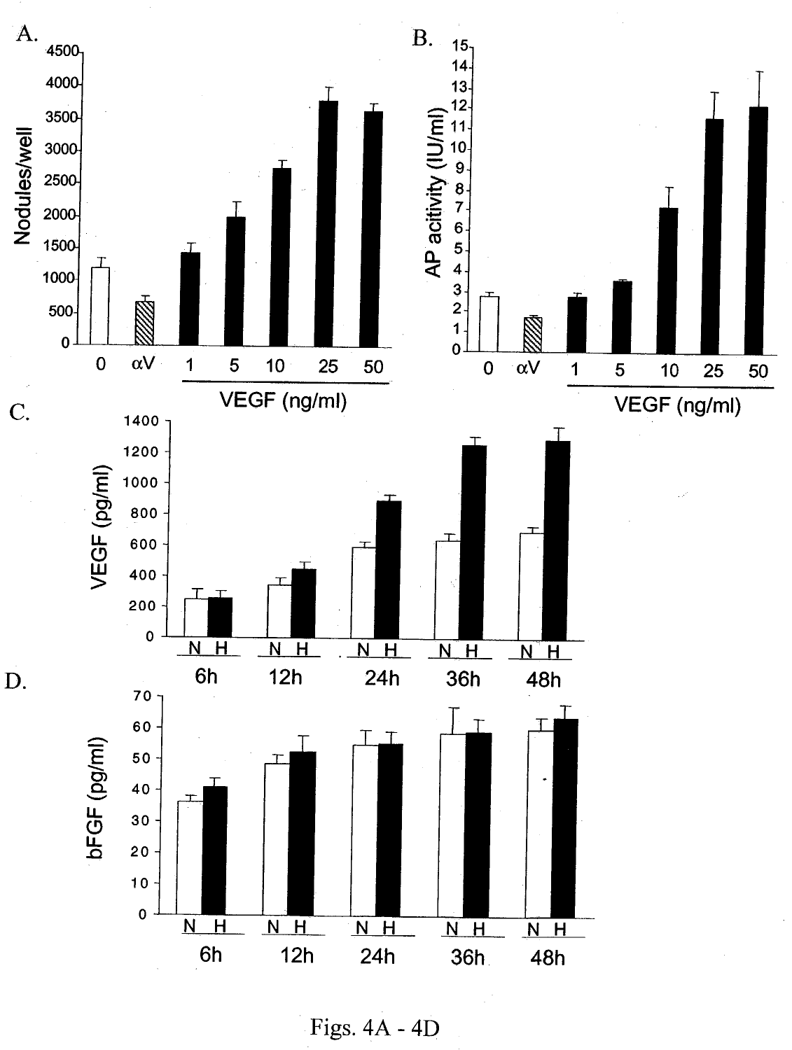

VEGF increased nodule formation and alkaline phosphatase activity (Midy and Plouet (1994) Biochem. Biophys. Res. Commun. 199:380-386) in a dose dependent manner in primary human osteoblasts (FIG. 4A), which may be more sensitive to VEGF than a mouse pre-osteoblast cell lines. Expression of VEGF receptors in our primary osteoblasts appeared to be widespread, as ˜98% of cells bound biotinylated VEGF. Unlike a mouse pre-osteoblast cell line as used in Deckers et al. (2000) Endocrinology 141:1667-1674, primary human osteoblasts responded to inhibition of VEGF (via a VEGF neutralizing antibody (0.3 μg/mL)) with a 43.3% decrease in nodule formation and a 39.3% decline in alkaline phosphatase production (FIG. 4b). VEGF, but not bFGF, was up-regulated by 96.6% in these cells in vitro (FIG. 4c, 4d) under hypoxic conditions (Steinbrech et al. (2000) Am. J Physiol. Cell. Physiol. 278:C853-C860). Thus, hypoxia-induced upregulation of VEGF in osteoblasts at a site of injury may contribute to local osteoblast activation and promote initial calcification of the fracture hematoma.

### Example 3

### Local VEGF Treatment Promotes Bone Repair

Treatment with a Slow Release Formulation of VEGF

To test the effect of VEGF on repair of femur fractures, a standard, stabilized, mid-shaft femur fracture was created (as described above) and included disruption of the periosteum in order to model a challenged fracture in humans. Bio-erodible polylactic acid (PLA) depot (PLAD) combines PLA (Resomer® R 202H, Boehringer Ingleheim, Ingleheim, Germany) with hydrophilic (benzyl alcohol, BA) and hydrophobic (benzyl benzoate, BB) solvents, to dissolve protein and solvate PLA, respectively. Cleland et al. (2001) J. Contr. Rel. 72:13-24. Liquid recombinant murine vascular endothelial growth factor (muVEGF) (Genentech), was spray freeze-dried to produce a powder for formulation as a solid phase in the PLAD system. Based on the findings that rates of release were inversely related to the percent PLA (data not shown), 40% PLA was used. PLAD solutions were prepared from a mixture of 40% (w/w) PLA, 5% (w/w) BA, (low peroxide, double distilled USP grade, Genentech) and 55% (w/w) BB (Sigma, USP grade). PLAD muVEGF was made by homogenizing (5 mm microfine shear homogenizer, VirTis) muVEGF powder with this PLAD solution for 2 minutes at 8000 rpm. On injection, the PLAD solution formed a soft depot for protein release over time. This system allowed for a local, low viscosity dosing solution with a high protein drug dose in a small volume. For each fractured mice, 10 μL of the PLAD with or without VEGF (0-10 μg) was applied directly at the defect site.

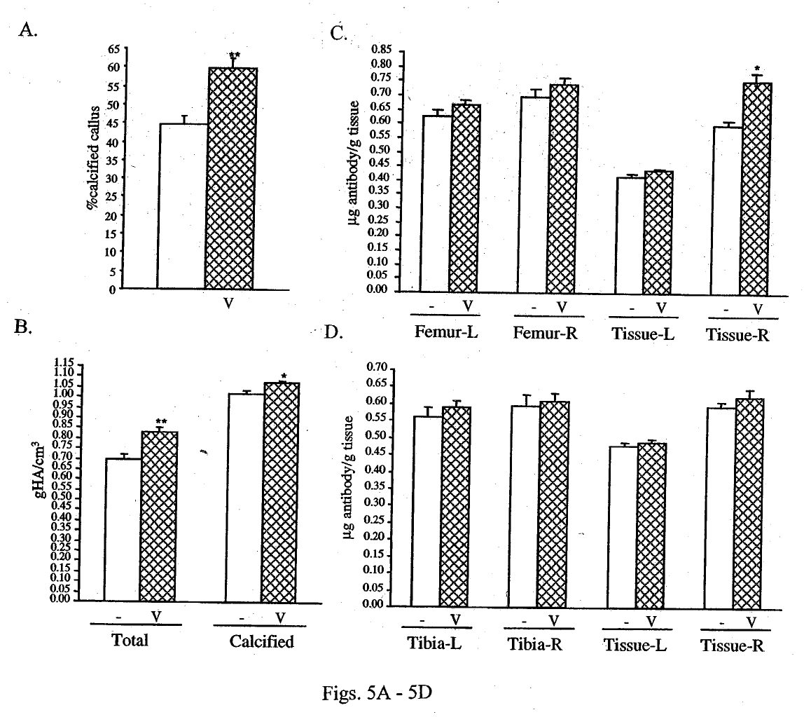

Treatment with slow-release VEGF (10 μg) caused a 33.6% increase (P=0.0001) in degree of mineralization (FIG. 5A), and augmented mean mineral density in the total callus by 19.2% (p=0.0001) and calcified callus by 5.2% (p=0.007) (FIG. 5B). As described in Example 1 (FIG. 3b), vascularity was increased in soft tissues in both the upper and lower legs on the fractured side (right) relative to contra-lateral, intact (left) legs in both control and VEGF-treated mice (FIGS. 5C and 5D). VEGF (10 μg) treatment further stimulated induction by 19.9% (P=0.0002) in the upper leg (the site of VEGF application) (FIG. 5C), but not in the lower leg (FIG. 5D). Thus, although the fracture itself increased vascularity in both the upper and lower part of the injured leg, VEGF treatment in the femur led to local enhancement of angiogenesis around the femur without affecting nearby soft tissues in the tibia.

A pharmacokinetic study using ELSA to measure systemic levels of VEGF indicated that local, sustained administration of the slow-release VEGF formulation did not elevate VEGF level in circulation. Exogenous VEGF was un-detectable in plasma 72 hours after delivery of VEGF to the fracture site. As such, little or none systemic effects of the locally administered VEGF are expected to the treated subject.

VEGF Stimulates Repair of a Critical Size Defect in Rabbit Radii

To test the therapeutic potential of VEGF in another species and model, critical size defects (a 10 mm gap) were created in rabbit radii and implanted a pump with various concentrations of VEGF continuously released over the first 7 days after surgery. In 30 (6 animals/group) anesthetized, male NZW rabbits, the interosseous ligament was separated, and the periosteum was excised from the radius 1.2 cm along the mid-shaft. A sterile spatula was placed between the radius and the ulna, and a 1 cm segment of the radius was removed using a sterile saw blade attached to an electric drill, with liberal irrigation with saline to prevent overheating of bone margins. A local, subcutaneous osmotic pump (Alzet model 2001, 1 μl/hr) was used to continuously release VEGF (0, 50, 100, 250, and 1000 ug) over the first 7 days after surgery. Analgesics were given prior to surgery (0.02-0.1 mg/kg subcutaneous buprenorphine) and for 72 h post-surgery (fentanyl transdermal patch (25 μg/hr)).

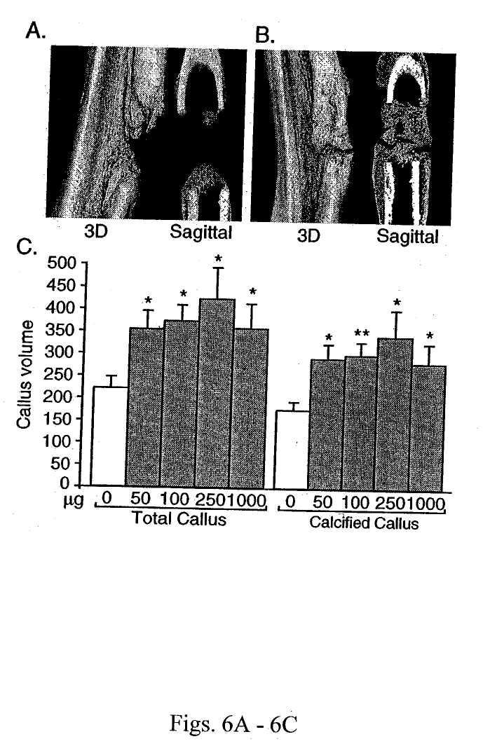

X-ray and CT analyses at 28 days showed that, while placebo-treated defects were not able to create a bony bridge across the gap (FIG. 6A), VEGF treatment caused filling of the defect with ossified bone (FIG. 6B). At the most effective dose tested (250 μg), VEGF caused a 23% increase (P=0.02) in total callus volume and a 27.5% increase (P=0.02) in calcified callus volume (FIG. 6C). VEGF at the highest concentration (1000 μg) was less potent than VEGF 250 μg (FIG. 6C).

Old Mice with Bone Defects Respond Positively to VEGF Treatment

Young and old mice with bone defects were compared for their recovery with or without exogenous VEGF. A focal cortical defect in the tibia was created in each mice according to the procedures described in Example 1. In a subset of old mice group, VEGF was applied to the site of the defect before the tibia specimen was dissected and examined for repair. While young mice with tibia defect had a quicker recovery, older mice did show significant improvement in recovery when exogenous VEGF was introduced at the site. In a fracture repair experiment, older mice treated with VEGF showed significant increase in bone regeneration at the site of the injury when compared with old, injured mice without VEGF treatment.

In a vascularity experiment similar to the one described in Example 1 concerning Fit-IgG treated animals, young and old mice were directly compared for their basal vascularity levels and vascularity changes over time after incurring fracture. Young mice in general had higher basal levels of vascularity than older mice. After fracture, young mice showed significant, time-independent increase of vascularity due to angiogenesis induction as described above, whereas old mice responded to fracture with much less, slower increase of vascularity.

Taken together, our results indicate that older animals, which have lower vascularity and arguably poorer regeneration capability, can respond positively to a VEGF treatment for bone repair.

### Example 4

### Receptor-Selective VEGF Variants Promote Bone Repair In Vivo

VEGF variants with selective binding activity to either KDR or Flt receptor were used to test their effects on repair of femur fractures in vivo. Standard, stabilized, mid-shaft femur fractures (as described above in Example 1) were created in mice and included disruption of the periosteum in order to model a challenged fracture in humans. Bio-erodible polylactic acid (PLA) depot (PLAD) combines PLA (Resomer® R 202H, Boehringer Ingleheim, Ingleheim, Germany) with hydrophilic (benzyl alcohol, BA) and hydrophobic (benzyl benzoate, BB) solvents, to dissolve protein and solvate PLA, respectively. Cleland et al. (2001) J. Contr. Rel. 72:13-24. The wild type VEGF, a KDR-selective VEGF variant with mutations D63S/G65M/L66R, and a Flg-selective VEGF variant with mutations 143A/146A/Q79A/183A were used as treatment agents in this study. Li et al. (2000) J. Biol. Chem. 275:29823-29828. To prepare the slow-release formulation for local delivery, VEGF or VEGF variant in liquid form was spray freeze-dried to produce a powder for formulation as a solid phase in the PLAD system. Based on the findings that rates of release were inversely related to the percent PLA (data not shown), 40% PLA was used. PLAD solutions were prepared from a mixture of 40% (w/w) PLA, 1% (w/w) BA, (low peroxide, double distilled USP grade, Genentech) and 59% (w/w) BB (Sigma, USP grade). PLAD muVEGF or PLAD-VEGF variant was made by homogenizing (5 mm microfine shear homogenizer, VirTis) muVEGF or VEGF variant powder with this PLAD solution for 2 minutes at 8000 rpm. On injection, the PLAD solution formed a soft depot for protein release over time. This system allowed for a local, low viscosity dosing solution with a high protein drug dose in a small volume. For each fractured mice, 10 μL of the PLAD alone or PLAD with 10 μg treatment agent was applied directly at the defect site.

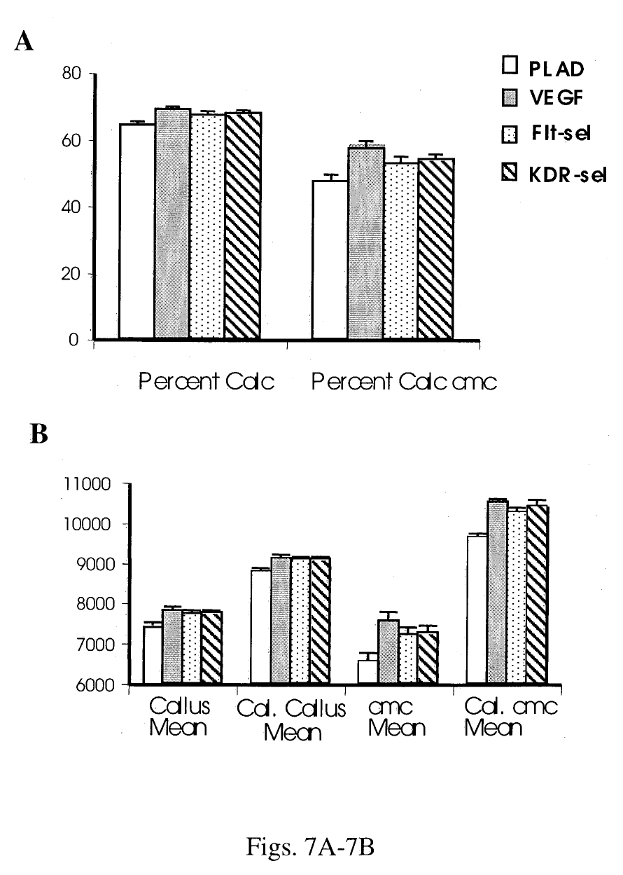

As shown in FIGS. 7A-7B, treatment with either KDR-selective or Flt-selective VEGF variant caused significant increase in percent calcified callus (FIG. 7A), and augmented mean mineral density in the total callus as well as in calcified callus in your mice (FIG. 7B). These results, showing significant differences with VEGF variants (Flt-sel or KDR-sel) relative to the control (PLAD carrier alone), indicate that VEGF variants can be useful to promote bone repair. Furthermore, these variants appear to be as active as the wild-type VEGF in young mice, raising the intriguing possibility of using VEGF variants (such as the Flt-sel variants) which effectively stimulate bone cells but are less active on endothelial cells. As such, if used as a therapeutic, such VEGF variant may cause less side effects in patients.

What is claimed is:

1. A method for promoting bone formation in a subject, comprising administering to the subject a composition comprising VEGF or variant thereof, in a manner effective to promote bone formation.

2. The method of claim 1, wherein the composition is administered through a local delivery system.

3. The method of claim 2, wherein the composition is in a slow release preparation.

4. The method of claim 3, wherein the slow release preparation comprises polylactic acids in combination with a hydrophilic solvent and a hydrophobic solvent.

5. The method of claim 4, wherein the hydrophilic solvent is benzyl alcohol.

6. The method of claim 4, wherein the hydrophobic solvent is benzyl benzoate.

7. The method of claim 4, wherein the slow release preparation is a PLAD comprising polylactic acid in combination with benzyl alcohol and benzyl benzoate.

8. The method of claim 1, wherein the composition comprising VEGF or variant thereof is administered in serial or in combination with another osteogenic agent.

9. The method of claim 8, wherein the osteogenic agent is selected from the group consisting of BMPs, FGF, growth hormone, PDGF, TGF-β, GDF-5 and OP-1.

10. A method for promoting bone repair in a subject having a bone defect, comprising administering to the subject a composition comprising VEGF or variant thereof, in a manner effective to repair the defect.

11. The method of claim 10, wherein the bone defect is associated with bone damage, acquired bone disorder or inheritable bone disease.

12. The method of claim 11, wherein the bone defect is nonunion.

13. The method of claim 11, wherein the bone defect is delayed union of the fracture.

14. The method of claim 10, wherein the bone defect is a damage in tendon, ligament, meniscus or cartilage.

15. The method of claim 10, wherein the bone defect is associated with verterbral body or disc injury.

16. The method of claim 10, wherein the bone defect is associated with avascular necrosis.

17. The method of claim 10, wherein the bone defect is a cranio-facial defect.

18. The method of claim 11, wherein the bone disorder is osteoporosis, osteoarthritis or osteosclerosis.

19. The method of claim 10, wherein the composition is administered through a local delivery system.

20. The method of claim 19, wherein the composition is in a slow release preparation.

21. The method of claim 20, wherein the slow release preparation comprises polylactic acids in combination with a hydrophilic agent and a hydrophobic agent.

22. The method of claim 21, wherein the hydrophilic agent is benzyl alcohol.

23. The method of claim 21, wherein the hydrophobic agent is benzyl benzoate.

24. The method of claim 21, wherein the slow release preparation is a PLAD comprising polylactic acid in combination with benzyl alcohol and benzyl benzoate.

25. The method of claim 10, wherein the composition is administered in serial or in combination with an agent or means facilitating the repair of the defect.

26. The method of claim 25, wherein the agent is an osteogenic agent selected from the group consisting of BMPs, FGF, growth hormone, PDGF, TGF-β, GDF-5 and OP-1.

27. The method of claim 25, wherein the means is a mechanical fixture at the site of the defect.

28. A slow release composition for promoting bone repair comprising: a) polylactic acid; b) at least one solvent; and c) VEGF or variant thereof.

29. The slow release composition of claim 28, where the solvent is ethanol, benzyl benzoate, miglyol, propylene carbonate, benzyl alcohol, ethyl lactate, glycofurol, N-methylpyrrolidone, a-pyrrolidone, propylene glycol, acetone, methyl acetate, ethyl acetate, methyl ethyl ketone, triacetin, dimethylformamide, dimethylsulfoxide, tetrahydrofuran, caprolactam, decylmethylsulfoxide, oleic acid, or 1-dodecyazacycloheptan-2-one.

30. The slow release composition of claim 28, where the solvent is a combination of benzyl alcohol and benzyl benzoate.

31. An article of manufacture comprising a) a container and a composition of claim 28 contained therein; and b) instruction for using said composition.
